(19) United States
(12) Patent Application Publication
(10) Pub. No.: US 2024/0268681 A1
(43) Pub. Date: Aug. 15, 2024

(54) TEMPERATURE MEASURING DEVICE AND TEMPERATURE MEASURING METHOD

(71) Applicant: JAPAN PRECISION INSTRUMENTS INC., Shibukawa-shi, Gunma (JP)

(72) Inventor: Tokurin SHOU, Shibukawa-shi (JP)

(73) Assignee: JAPAN PRECISION INSTRUMENTS INC., Shibukawa-shi, Gunma (JP)

(21) Appl. No.: 18/022,854

(22) PCT Filed: Sep. 29, 2022
(86) PCT No.: PCT/JP2022/036561

§ 371 Date: Feb. 23, 2023

(30) Foreign Application Priority Data
Jan. 17, 2022 (JP) 2022-004893

(51) Int. Cl. A61B 5/01; A61B 5/00; A61G 11/00

(52) U.S. Cl. CPC A61B 5/01; A61B 5/0059; A61G 11/00; A61B 2503/045

(57) ABSTRACT

A technique that enables accurate temperature measurement by detecting a relative positional deviation between a measurement object and a temperature sensor. A temperature measuring device, includes: a temperature sensor that measures a temperature of a measurement object in a non-contact manner with the measurement object; a light emitting element that emits light toward the measurement object; a light receiving element that receives reflected light of the light emitted from the light emitting element; and a control unit that detects a relative positional deviation between the measurement object and the temperature sensor based on a received light intensity by the light receiving element.

### TECHNICAL FIELD

The present disclosure relates to a temperature measuring device and a temperature measuring method.

### DESCRIPTION OF RELATED ART

In recent years, a non-contact temperature sensor has been used in various fields. As one aspect thereof, it has been proposed to measure a body temperature of a newborn baby or an infant housed in an incubator in a non-contact manner (see, for example, Patent document 1).

### PRIOR ART DOCUMENT

### Patent Document

[Patent Document 1] JP-A-2019-93143

### SUMMARY OF THE DISCLOSURE

### Problem to be Solved by the Disclosure

In a non-contact temperature sensor, a relative positional relationship (for example, each inter-distance) with respect to a measurement object is very important in ensuring measurement accuracy. However, when the measurement object is a newborn baby, an infant, or an animal, the measurement object may move even during measurement of a temperature. Therefore, due to a movement of the measurement object, there is a risk that a correct measurement result cannot be obtained due to deterioration in measurement accuracy, or that an incorrect measurement result may be obtained by measuring a portion other than the measurement object.

An object of the present disclosure is to provide a technique that enables an accurate temperature measurement by detecting a relative positional deviation between a measurement object and a temperature sensor, when performing temperature measurement using a non-contact temperature sensor.

### Means for Solving the Problem

According to an aspect of the present disclosure, there is provided a temperature measuring device, including:
a temperature sensor that measures a temperature of a measurement object in a non-contact manner with the measurement object;
a light emitting element that emits light toward the measurement object;
a light receiving element that receives reflected light of the light emitted from the light emitting element; and
a control unit that detects a relative positional deviation between the measurement object and the temperature sensor based on a received light intensity by the light receiving element.

Further, according to another aspect of the present disclosure, there is provided a temperature measuring method, including:
measuring a temperature of a measurement object using a non-contact temperature sensor;
emitting light from a light emitting element toward the measurement object and receiving reflected light of the light emitted from the light emitting element, with a light receiving element; and
detecting a relative positional deviation between the measurement object and the temperature sensor based on a received light intensity by the light receiving element.

### Advantage of the Disclosure

According to the present disclosure, when performing temperature measurement using a non-contact temperature sensor, it is possible to detect a relative positional deviation between the measurement object and the temperature sensor. Therefore, it is possible to prevent in advance a situation that a correct measurement result cannot be obtained or an incorrect measurement result is obtained due to the relative positional deviation, and as a result, it becomes possible to perform temperature measurement with high accuracy.

### BRIEF DESCRIPTION OF THE DRAWINGS

FIG. 1 is a block diagram schematically illustrating a functional configuration example of a temperature measuring device according to one embodiment of the present invention.

FIG. 2 is a plan view illustrating an example of a sensor arrangement when viewed from a side of a measurement object of the temperature measuring device according to one embodiment of the present invention.

FIG. 3 is a flow chart illustrating a specific example of a procedure of a temperature measuring method according to one embodiment of the present invention.

### DETAILED DESCRIPTION OF THE DISCLOSURE

A temperature measuring device and a temperature measuring method according to the present invention will be described below, with reference to the drawings.

### 1. Configuration of a Temperature Measuring Device

First, a configuration example of a temperature measuring device will be described.

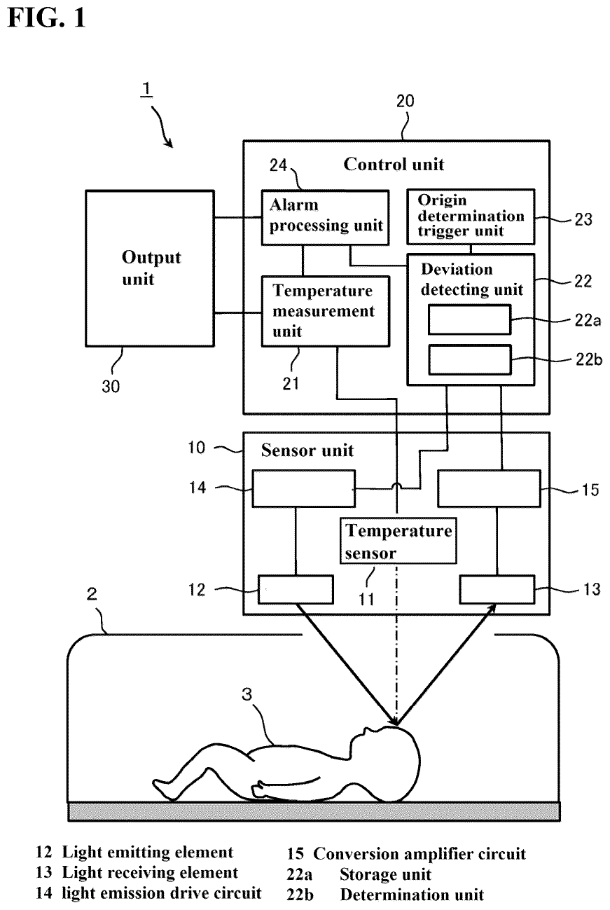

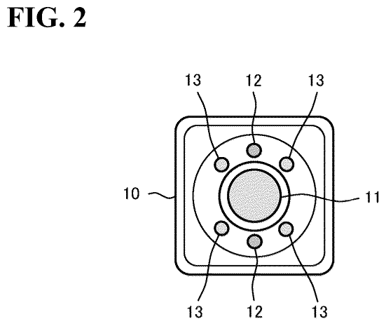

FIG. 1 is a block diagram schematically illustrating a functional configuration example of a temperature measuring device according to the present embodiment. FIG. 2 is a plan view illustrating an example of a sensor arrangement when viewed from a side of a measurement object of the temperature measuring device according to the present embodiment.

### (Overall Structure)

As illustrated in FIG. 1, a temperature measuring device 1 described as an example in the present embodiment is used to measure a temperature (specifically a body temperature obtained based on a skin temperature) of a human body such as a newborn baby or an infant housed in an incubator 2, or an animal equivalent thereto (hereinafter collectively referred to as “a measurement object”) 3.

That is, in the present embodiment, the temperature measuring device 1 is used in conjunction with the incubator 2. Then, in order to measure the temperature of the measurement object 3 in the incubator 2, the temperature measuring device 1 is broadly divided into a sensor unit 10, a control unit 20, and an output unit 30.

### (Sensor Unit)

The sensor unit 10 has a unit housing arranged so as to be able to adjust a relative positional relationship with the measurement object 3 in the incubator 2. Then, a temperature sensor 11, a light emitting element 12, a light receiving element 13, a light emission drive circuit 14, and a conversion amplifier circuit 15 are provided in the unit housing.

The temperature sensor 11 measures the temperature of the measurement object 3 in a non-contact manner with the measurement object 3. As such a temperature sensor 11, for example, it is conceivable to use an infrared sensor that performs temperature measurement using infrared rays. More specifically, it is conceivable to use a device that has a sensor detection surface arranged to face the measurement object 3, and that is configured to output an electric signal corresponding to an amount of infrared energy incident on the sensor detection surface. However, as long as a non-contact temperature measurement can be performed, the sensor is not necessarily limited to the infrared sensor, and a sensor using other measurement principle may also be used.

The light emitting element 12 emits light from within a unit housing of the sensor unit 10 toward the measurement object 3. The light emitted from the light emitting element 12 is, for example, visible light, infrared light, or ultraviolet light, and is not particularly limited as long as it has a wavelength that can be received by the light receiving element 13. As such a light emitting element 12, for example, it is conceivable to use an LED (light emitting diode) light emitting element.

Further, the light emitting element 12 is arranged near a sensor detection surface of the temperature sensor 11. That is, the light emitting element 12 is attached at a position near the temperature sensor 11. At least one light emitting element 12 may be arranged, but a plurality of (for example, two) light emitting element 12 may also be arranged to surround the sensor detection surface of the temperature sensor 11.

The light receiving element 13 receives reflected light of the light emitted from the light emitting element 12 within the unit housing of the sensor unit 10. As such a light receiving element 13, for example, a PD (Photodiode) light receiving element may be used, but the light receiving element is not limited thereto, and other light receiving element such as a phototransistor may also be used.

Further, the light receiving element 13 is arranged near the sensor detection surface of the temperature sensor 11 in the same manner as the light emitting element 12. That is, the light receiving element 13 is attached at a position near the temperature sensor 11. At least one light receiving element 13 may be arranged, but a plurality of (for example, four) light receiving element 13 may also be arranged to surround the sensor detection surface of the temperature sensor 11.

Specifically, for example FIG. 2 illustrates an arrangement example of the temperature sensor 11, the light emitting element 12, and the light receiving element 13. FIG. 2 is a plan view of the sensor unit 10 when viewed from the side of the measurement object 3, and is a view illustrating an example of a sensor arrangement on the surface of the measurement object 3 side.

As illustrated in the figure, on the surface of the measurement object 3 side in the sensor unit 10, the temperature sensor 11 is arranged so that the sensor detection surface of the temperature sensor 11 is positioned substantially in the center of the surface. Then, two light emitting elements 12 and four light receiving elements 13 are arranged to surround the sensor detection surface of the temperature sensor 11.

Each of the two light emitting elements 12 is evenly arranged around the sensor detection surface of the temperature sensor 11. For example, when an upper side in FIG. 2 is 0° direction, the light emitting elements 12 are arranged in 0° direction and 180° direction respectively.

Four light receiving elements 13 are evenly arranged around the sensor detection surface of the temperature sensor 11 in the same manner as the light emitting elements 12. Thereby, the light receiving elements 13 are arranged on all four sides respectively when viewed from the temperature sensor 11. The four sides here mean that the light receiving elements 13 are arranged in 45°, 135°, 225°, and 3150 directions respectively when the upper side in FIG. 2 is 0° direction, for example.

However, numerical values of the angles given here are merely one specific example, and the arrangement of the light emitting elements 12 and the light receiving elements 13 is not limited to these numerical values.

The light emitting elements 12 and the light receiving elements 13 are arranged apart from each other by a predetermined distance (for example, 0.2 to 1.5 cm, preferably about 0.8 cm) or more, or a light shielding member (for example, a plate-shaped member having no light transparency) is arranged between them. This is to prevent the light emitted from the light emitting elements 12, from directly entering the light receiving elements 13.

Further, in FIG. 1, the light emission drive circuit 14 is an electronic circuit for driving the light emitting element 12 to cause the light emitting element 12 to emit light. In the present embodiment, a case where the light emission drive circuit 14 is provided inside the unit housing of the sensor unit 10 will be taken as an example, but the present embodiment is not necessarily limited thereto, and the light emission drive circuit 14 may also be provided in a control unit 20, which will be described later.

The conversion amplifier circuit 15 is an electronic circuit for converting a received light intensity of a reflected light received by the light receiving element 13 into an electric signal, and for amplifying and outputting the electric signal. In the present embodiment, in the same manner as the light emission drive circuit 14, a case where the conversion amplifier circuit 15 is provided in the unit housing of the sensor unit 10 is taken as an example, but the present embodiment is not necessarily limited thereto, and the conversion amplifier circuit 15 may also be provided in the control unit 20 described later.

### (Control Unit)

The control unit 20 is for controlling a processing operation of the temperature measuring device 1, and is configured with a hardware resource including a combination of a CPU (Central Processing Unit), various memory devices, and the like. That is, the control unit 20 is configured with a hardware resource as a microcomputer, and by execution by CPU of a program stored in a memory, the program (software) and the hardware resource cooperate to control the processing operation of the temperature measuring device 1.

Further, the control unit 20 functions at least as a temperature measurement unit 21, a deviation detecting unit 22, an origin determination trigger unit 23, and an alarm processing unit 24 by executing the program by CPU.

The temperature measurement unit 21 receives an electric signal that is a result of detection by the temperature sensor 11, performs arithmetic processing based on the electric signal, and obtains a temperature value that is a result of measuring the temperature of the measurement object 3. The arithmetic processing performed by the temperature measurement unit 21 includes: for example an arithmetic processing for converting an amount of infrared incident energy into a temperature value, and an arithmetic processing for deriving a brain temperature (body temperature) from a skin temperature of the measurement object 3 based on preset relational data, and the like.

A deviation detecting unit 22 detects a relative positional deviation between the measurement object 3 and the temperature sensor 11 based on the received light intensity by the light receiving element 13. More specifically, the deviation detecting unit 22 receives the electric signal obtained by the conversion amplifier circuit 15 converting and amplifying the received light intensity by the light receiving element 13, and detects presence or absence of the relative positional deviation between the measurement object 3 and the temperature sensor 11 based on the electrical signal regarding the received light intensity. Therefore, the deviation detecting unit 22 functions as a storage unit 22a and a determination unit 22b.

The storage unit 22a stores the received light intensity of the light received by the light receiving element 13 when the measurement object 3 is positioned at a predetermined point, as an origin received light intensity. Details of the predetermined point will be described later.

The determination unit 22b compares the origin received light intensity and the received light intensity obtained by the light receiving element 13, with the origin received light intensity stored in the storage unit 22a as a reference, and when a difference between them becomes equal to or greater than a predetermined threshold value, it is determined that the positional deviation of the measurement object 3 has occurred. Details of the predetermined threshold value and a specific method of determination will be described later.

An origin determination trigger unit 23 generates an origin determination trigger to specify the origin received light intensity and stores it in the storage unit 22a. Details of the origin determination trigger and the origin received light intensity will be described later.

When the determination unit 22b determines that the positional deviation of the measurement object 3 has occurred, the alarm processing unit 24 performs alarm processing in response to the determination result. The alarm processing includes, for example, information output processing for notifying that the positional deviation of the measurement object 3 has occurred.

### (Output Unit)

The output unit 30 includes a display device such as a liquid crystal display, a communication device for communicating with an external device, etc., and outputs information to a user of the temperature measuring device 1 as needed. Information output by the output unit 30 includes: for example, information about a temperature value obtained by the temperature measurement unit 21, notification information (alarm information) from the alarm processing unit 24, etc.

### 2. Temperature Measurement Procedure

Next, an example of a processing operation by the temperature measuring device 1 having the above-described configuration, that is, the procedure of a temperature measuring method according to the present embodiment will be described.

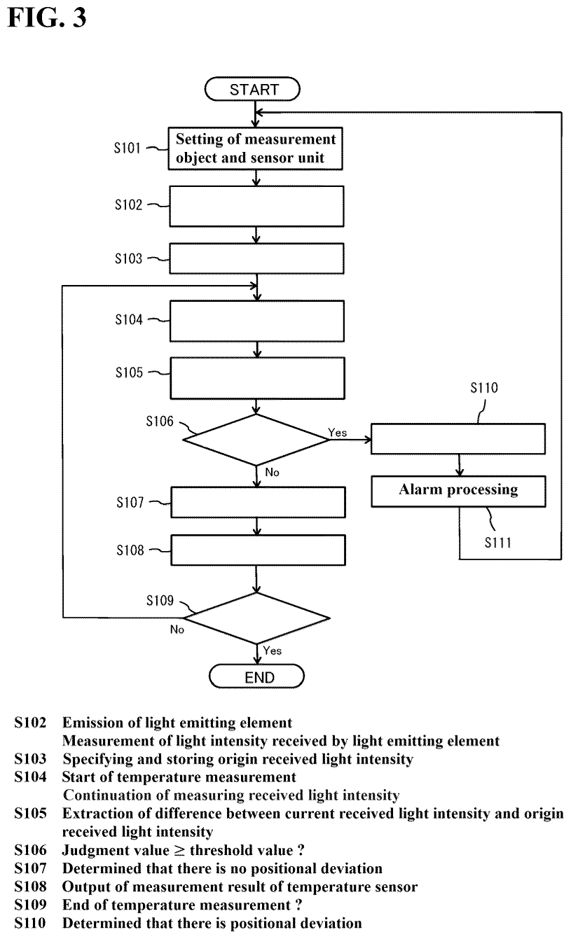

FIG. 3 is a flow chart showing a specific example of a procedure of the temperature measuring method according to the present embodiment.

As illustrated in FIG. 3, when using the temperature measuring device 1, first, the measurement object 3 and the sensor unit 10 are set (step 101, hereinafter step is abbreviated as “S”). Specifically, the measurement object 3 is housed in the incubator 2. Then, the sensor unit 10 attached to the incubator 2 is fixed by adjusting the relative positional relationship with the measurement object 3, so that the temperature sensor 11 of the sensor unit 10 faces a measurement point (for example, a forehead) of the measurement object 3 in the incubator 2, and so as to be positioned at a distance where temperature measurement is possible from the measurement point. Thereby, the relative positional relationship between the sensor unit 10 and the measurement object 3 is established, and therefore the temperature sensor 11 of the sensor unit 10 becomes capable of measuring the temperature of the measurement object 3 in a non-contact manner with the measurement object.

After setting the measurement object 3 and the sensor unit 10, in the sensor unit 10, the light emitting element 12 emits light toward the measurement object 3 in the incubator 2, and the light receiving element 13 receives the light reflected by the measurement object 3 (S102). Then, a conversion amplifier circuit 15 converts and amplifies the received light intensity of the reflected light received by the light receiving element 13 into an electric signal, and outputs it to the control unit 20. Thereby, the control unit 20 can measure the received light intensity of the light received by the light receiving element 13 based on the converted and amplified electrical signal.

When the measurement object 3 is a newborn baby, an infant, an animal, etc., the measurement object 3 may move even though the temperature is being measured. In such a case, even though the temperature sensor 11 can measure the temperature of the measurement object 3, the movement of the measurement object 3 causes a deviation in the relative positional relationship between the temperature sensor 11 and a point to be measured of the measurement object 3, and therefore there is a risk that a correct measurement result may not be obtained due to deterioration in measurement accuracy, or that an incorrect measurement result may be obtained by measuring a portion other than the measurement object.

Therefore, in the present embodiment, after the relative positional relationship between the sensor unit 10 and the measurement object 3 is determined, the origin determination trigger unit 23 generates an origin determination trigger, and in response to this origin determination trigger, the origin received light intensity is specified and stored (S103).

The origin determination trigger serves as a trigger for specifying and storing the origin received light intensity, and the origin determination trigger unit 23 generates the origin determination trigger at the timing described below.

For example, as a first trigger generation mode, the following case is considered. The temperature sensor 11 of the sensor unit 10 is configured to emit light (such as a visible light that can be visually recognized by a user) for indicating a position of a measurement point. In such a case, the origin determination trigger is generated in conjunction with ON/OFF control for the light for indicating the position. Specifically, when the relative positional relationship between the sensor unit 10 and the measurement object 3 is determined, thereafter, the timing comes when the temperature sensor 11 emits the light for indicating the position (that is, ON control is given for the light), and therefore at that timing, the origin determination trigger is generated.

Further, for example, as a second trigger generation mode, the following case is considered. The sensor unit 10 has a movement detection function using an acceleration sensor, etc. In such a case, when the relative positional relationship between the sensor unit 10 and the measurement object 3 is determined (that is, the movement of the sensor unit 10 is completed), this is detected by the movement detection function, and therefore at such a detection timing (that is, the movement completion timing of the sensor unit 10), the origin determination trigger is generated.

Further, for example, as a third trigger generation mode, the following case is considered. An operation unit operated by a user of the temperature measuring device 1 and a communication unit for communicating with a host device, etc. are connected to the control unit 20. In such a case, when a predetermined operation for generating a trigger is performed by the operation unit, or when a predetermined signal for generating a trigger is transmitted from a host device, in response to these, the origin determination trigger is generated at the timing when a predetermined operation is performed, and at the timing when a predetermined signal is received, or the like.

Then, depending on the generation of the origin determination trigger, the origin determination trigger unit 23 specifies the received light intensity of the reflected light received by the light receiving element 13 at that time point, as the origin received light intensity. That is, the origin received light intensity corresponds to a result of measuring the received light intensity by the light receiving element 13 at the time point when the origin determination trigger is generated.

The specified origin received light intensity is stored and held in the storage unit 22a until the origin received light intensity is reset. That is, the storage unit 22a stores the received light intensity of the light received by the light receiving element 13 when the measurement object 3 is positioned at a predetermined point, as the origin received light intensity. The predetermined point here is a point where the measurement object 3 is positioned when the origin determination trigger is generated, and this is the point where the measurement object 3 is positioned when the relative positional relationship between the sensor unit 10 and the measurement object 3 is determined.

After specifying and storing the origin received light intensity, the temperature measurement for the measurement object 3 is started using the temperature sensor 11, and the temperature measurement is continuously performed. Then, after the temperature measurement is started, the light emission from the light emitting element 12 and the measurement of the received light intensity by the light receiving element 13 are continuously performed in parallel with the temperature measurement (S104). Here, “continuously” means that measurements are repeatedly performed at preset regular timings (for example, every 100 milliseconds to several tens of seconds). The light emission from the light emitting element 12 may be performed at a constant light emission intensity so as to continue a light emission state.

At this time, the deviation detecting unit 22 of the control unit 20 recognizes the received light intensity as a current received light intensity, every time the measurement result of the received light intensity by the light receiving element 13 (that is, the electric signal from the conversion amplifier circuit 15) is received. That is, the current received light intensity corresponds to the measurement result of the received light intensity by the light receiving element 13 at each time point while performing temperature measurement by the temperature sensor 11.

Then, when the current received light intensity is recognized, the deviation detecting unit 22 reads out the origin received light intensity as a reference, from the storage unit 22a, and compares the origin received light intensity with the current received light intensity obtained from the light receiving element 13, and these differences are extracted and used as judgment values for judging presence or absence of generation of the positional deviation (S105). That is, based on the current received light intensity and the origin received light intensity, the determination unit 22b of the deviation detecting unit 22 extracts the judgment values using the following arithmetic expression.

Judgment value=|current received light intensity−origin received light intensity|

When extracting the judgment values, further, the determination unit 22b of the deviation detecting unit 22 compares the judgment values (that is, a difference between the current received light intensity and the origin received light intensity) with a predetermined threshold value set in advance, and determines whether or not the difference is equal to or greater than the threshold value (S106). The predetermined threshold value corresponds to a size of an allowable difference (judgment value), and the size of the value is not particularly limited as long as it is set in advance.

As a result, when the judgment value is less than the threshold value, that is, the judgment value < the threshold value (S106: No), the difference between the current received light intensity and the origin received light intensity does not exceed the allowable range, and therefore the determination unit 22b determines that the position of the measurement object 3 has not deviated (S107). That is, the determination unit 22b determines that the measurement object 3 has not moved enough to interfere with temperature measurement by the temperature sensor 11, and notifies the temperature measurement unit 21 of this matter, to allow the temperature sensor 11 to continue the temperature measurement for the measurement object 3.

Thereby, the result of the temperature measurement by the temperature sensor 11 is processed by the temperature measurement unit 21 and then outputted as information from the output unit 30 (S108). As a result, a user of the temperature measuring device 1 can recognize a temperature value (body temperature) of the measurement object 3 in the incubator 2 through an output content of the output unit 30. The information output from the output unit 30 is continuously performed until the temperature sensor 11 finishes the temperature measurement.

Thereafter, the control unit 20 determines whether or not to end the temperature measurement by the temperature sensor 11 (S109). This determination may be made according to, for example, whether or not there is a predetermined operation by the operation unit, or whether or not there is a transmission of a predetermined signal from the host device. Then, from the step (S104), the control unit 20 repeats the above-described steps (S104 to S109) for starting temperature measurement by the temperature sensor 11, until it is determined to end the temperature measurement.

On the other hand, as a result of a comparison between the judgement value and the predetermined threshold value, when the judgement value ≥ threshold value (S106: Yes), there is a difference greater than the allowable range between the current received light intensity and the origin received light intensity. Therefore, the determination unit 22b determines that the positional deviation of the measurement object 3 has occurred (S110). That is, the determination unit 22b determines that the movement of the measurement object 3 may hinder the temperature measurement by the temperature sensor 11, and notifies the alarm processing unit 24 of this matter.

Upon receiving this notification, the alarm processing unit 24 performs alarm processing in response to the determination result by the determination unit 22b (S111). Specifically, as the alarm processing, the alarm processing unit 24 notifies the temperature measurement unit 21 that the positional deviation of the measurement object 3 has occurred, for example, and interrupts the temperature measurement for the measurement object 3 by the temperature sensor 11. Further, as the alarm processing, the alarm processing unit 24 causes the output unit 30 to output, for example, information for notifying that the positional deviation of the measurement object 3 has occurred. As a result, the user of the temperature measuring device 1 can recognize through the output content of the output unit 30 that the temperature measurement for the measurement object 3 cannot be performed due to the movement of the measurement object 3 in the incubator 2.

When there is the alarm processing by the alarm processing unit 24, for example, by resetting the measurement object 3 and the sensor unit 10 by the user of the temperature measuring device 1 (S101), it becomes possible to perform temperature measurement for the measurement object 3 again.

As described above, in the present embodiment, using the light emitting element 12 and the light receiving element 13, it is determined whether or not the positional deviation of the measurement object 3 has occurred, based on the received light intensity by the light receiving element 13. Accordingly, it is possible to prevent in advance the occurrence of the following situation. A correct temperature measurement result cannot be obtained, or an incorrect temperature measurement result is obtained due to the relative positional deviation between the measurement object 3 and the temperature sensor 11. As a result, it becomes possible to perform temperature measurement with high accuracy.

Such a detection of the relative positional deviation between the measurement object 3 and the temperature sensor 11 can also be performed, for example, by providing an imaging camera separately from the sensor unit 10 and analyzing image data obtained by the imaging camera. However, in this case, since the imaging camera is required, there is a problem that a device configuration becomes complicated, and a data processing load for analyzing the image data becomes excessive. In this regard, in the present embodiment, since the light emitting element 12 and the light receiving element 13 are used, the relative positional deviation can be detected with a very simple device configuration without complicating the device configuration. Further, since the data processing load does not become excessive, it is possible to ensure quick responsiveness in detecting the relative positional deviation.

### 3. Details of the Temperature Measurement

Next, details of the processing operation performed using the sensor unit 10 in the temperature measuring method of the procedure described above will be described more specifically.

### (Element Arrangement)

In the present embodiment, the sensor unit 10 includes a temperature sensor 11, a plurality of (for example two) light emitting elements 12 and a plurality of (for example four) light receiving elements 13, in addition to the temperature sensor 11. That is, even when a single light emitting element 12 and a single light receiving element 13 are arranged, it is possible to perform the temperature measurement method including the procedure described above. However, it is preferable that a plurality of light emitting elements 12 and light receiving elements 13 are arranged in the sensor unit 10, respectively. In this case, the number of the light emitting elements 12 and the light receiving elements 13 to be arranged, may not be the same number (that is, the ratio of 1:1).

For example, it is preferable that the light receiving elements 13 are evenly arranged at four points around the temperature sensor 11 so that each light receiving element 13 surrounds the temperature sensor 11 on all four sides. Specifically, in the arrangement mode illustrated in FIG. 2, the light receiving elements 13 are arranged in directions of 45°, 135°, 225°, and 3150 when an upper side in the figure is a direction of 0°.

In such an arrangement mode, for example, when the measurement object 3 moves toward 0° direction (overhead direction of the measurement object 3), the current received light intensity at each of the light receiving elements 13 in 450 direction and 315° direction increases. Further, for example, when the measurement object 3 moves toward 45° direction, the current received light intensity at the light receiving element 13 in 45° direction increases. Further, for example, when the measurement object 3 moves in 90° direction (toward a left hand side of the measurement object 3 in the supine posture), the current received light intensity at each of the light receiving elements 13 in 45° direction and 135° direction increases.

Accordingly, when a plurality of light receiving elements 13 are arranged, it becomes possible to not only determine whether or not the positional deviation of the measurement object 3 has occurred, but also determine the moving direction when the positional deviation occurs. Particularly, when the light receiving elements 13 are evenly arranged on all four sides of the temperature sensor 11, it is possible to determine a general direction of the movement of the measurement object 3 in any direction. This is the same even when an arrangement angle of each light receiving element 13 is different from that illustrated in FIG. 2, as long as the light receiving elements 13 are evenly arranged on all four sides of the temperature sensor 11.

When the moving direction of the measurement object 3 can be determined, by using a determination result, it is possible to easily and quickly reset the measurement object 3 and the sensor unit 10 in the event of the alarm processing. Further, by notifying the user of the determination result regarding the moving direction of the measurement object 3, it is possible to alert the user of the temperature measuring device 1.

Further, for example, it is preferable that the light emitting elements 12 are evenly arranged at two points around the temperature sensor 11. Specifically, in the arrangement mode illustrated in FIG. 2, the light emitting elements 12 are arranged to be positioned between the light receiving elements 13 surrounding the four sides of the temperature sensor 11, and in the arrangement mode illustrated in FIG. 2, the light emitting elements 12 are arranged in 0° direction and 180° direction respectively, when the upper side in the figure is 0° direction.

With such an arrangement mode, it is possible to suppress an unevenness in the received light intensity of each of the light receiving elements 13, even when the light receiving elements 13 are arranged on all four sides of the temperature sensor 11. That is, for example, when the light emitting element 12 is singular, a distance between the light emitting element 12 and each light receiving element 13 is not uniform, and there is a possibility that the received light intensity of each light receiving element 13 becomes uneven depending on a difference in the distance. However, in the arrangement mode illustrated in FIG. 2, such an unevenness can be suppressed, and as a result, a detection accuracy regarding the positional deviation of the measurement object 3 can be improved. In addition, since the number of the light emitting elements 12 is not the same as that of the light receiving elements 13 and the light emitting elements 12 are arranged only at two points, it is possible to suppress the complication of the configuration of the sensor unit 10 while suppressing the unevenness of the received light intensity of each light receiving element 13, and thereby the miniaturization of the sensor unit 10 can be easily realized.

### (Control of Light Emission)

For each of the light emitting elements 12 in such an arrangement mode, the light emission from the light emitting elements 12 is controlled by a light emission drive circuit 14 that is operated according to an instruction from the control unit 20. That is, control of the light emission from each light emitting element 12 is performed by the light emission drive circuit 14. The control of the light emission from the light emitting element 12 includes the following modes.

For example, from the light emitting element 12, light is emitted at a constant emission intensity while the light is emitted continuously. Thereby, stable light emission can be performed without flickering, etc. However, the light emission is not limited thereto, and for example, pulsed light emission with changing emission intensity may also be performed. That is, the light emission is performed in an intermittent mode (specifically, a mode in which the light emitting element 12 is blinked at a constant frequency at a high speed) or in a mode in which a strength of the light emission intensity is changed (specifically, in a mode in which the strength of the light emission intensity is switched at a high speed at a constant frequency). This makes it possible to extend a life of the light emitting element 12 while suppressing a power consumption. Further, by varying the pulse frequency and adjusting a length of the light emission time, it is also possible to control a brightness of the light from the light emitting element 12.

Further, it is conceivable that the light emission intensity of the light emitting element 12 is set in advance so that an influence of ambient light does not reach the received light intensity by the light receiving element 13. By setting the light emission intensity in this way, it is possible to prevent the brightness of an indoor environment from affecting the light intensity received by the light receiving element 13, and as a result, it becomes possible to appropriately detect the relative positional deviation of the measurement object 3, no matter what indoor environment the sensor unit 10 is used in.

Further, the light emitted from the light emitting element 12 may be the light having a wavelength that can be received by the light receiving element 13, such as visible light, infrared light, or ultraviolet light. However, when the emitted light is infrared light and an infrared sensor is used as the temperature sensor 11, it is preferable that the light has a wavelength that does not affect the temperature measurement by the temperature sensor 11. Thereby, the temperature measurement by the temperature sensor 11 can be performed with high accuracy.

### 4. Effect of the Present Embodiment

According to the present embodiment, one or more of the following effects can be obtained.

(a) In the present embodiment, when the temperature measurement is performed using the non-contact temperature sensor 11, the light emitting element 12 and the light receiving element 13 are used, and based on the received light intensity by the light receiving element 13, the relative positional deviation between the measuring object 3 and the temperature sensor 11 is detected. Accordingly, it is possible to prevent in advance the following situation. A correct temperature measurement result cannot be obtained or an incorrect temperature measurement result is obtained due to the relative positional deviation between the measurement object 3 and the temperature sensor 11.

In addition, in the present embodiment, the relative positional deviation is detected using the light emitting element 12 and the light receiving element 13. Therefore, the relative positional deviation can be detected with a very simple device configuration without complicating the device configuration, for example, unlike a case where image data is analyzed by an imaging camera. Further, a data processing load does not become excessive. Therefore, quick responsiveness can be ensured in detecting the relative positional deviation.

That is, according to the present embodiment, when performing temperature measurement using the non-contact temperature sensor 11, by detecting the relative positional deviation between the measurement object 3 and the temperature sensor 11, it is possible to perform the temperature measurement with a very simple configuration and with high accuracy.

(b) In the present embodiment, the received light intensity by the light receiving element 13 at the time of positioning the measurement object 3 at a predetermined position, is stored as the origin received light intensity, and when a judgment value, which is a difference between the origin received light intensity and the current received light intensity obtained by the light receiving element 13, becomes equal to or greater than a predetermined threshold value, it is determined that the position of the measurement object 3 has deviated. That is, the relative positional deviation between the measurement object 3 and the temperature sensor 11 is detected based on a state when the measurement object 3 is positioned at a predetermined point (that is, a state before the measurement object 3 moves).

Therefore, according to the present embodiment, by using the origin received light intensity as a reference, it is possible to reliably and accurately determine that the positional deviation of the measurement object 3 has occurred. In addition, the difference from the reference is compared with a predetermined threshold value to make a judgment. Therefore, depending on the setting of the predetermined threshold value, it becomes possible to determine whether or not the degree of the positional deviation of the measurement object 3 is such that the temperature of the measurement object 3 cannot be measured, which is also very effective in performing temperature measurement by the temperature sensor 11 with high accuracy.

(c) In the present embodiment, the origin received light intensity is specified in response to the origin determination trigger. Thereby, the received light intensity by the light receiving element 13 at the time of determining the relative positional relationship between the measurement object 3 and the sensor unit 10, can be used as the origin received light intensity, and it is possible to set a determination criteria for detecting the relative positional deviation in accordance with an actual use state of the incubator 2 and the temperature measuring device 1.

That is, according to the present embodiment, it is possible to reliably and accurately detect the relative positional deviation between the measurement object 3 and the temperature sensor 11, according to the actual use state of the incubator 2 and the temperature measuring device 1, which is highly convenient for the user of the temperature measuring device 1.

(d) In the present embodiment, the light emitting element 12 and the light receiving element 13 are attached to the temperature sensor 11. That is, the light emitting element 12 and the light receiving element 13 emit light and receive reflected light at a position near the temperature sensor 11. Therefore, according to the present embodiment, it is determined whether or not the relative positional deviation of the measurement object 3 has occurred when viewed from a position near the temperature sensor 11. Therefore, it becomes possible to appropriately determine whether or not there is a problem with the temperature measurement by the temperature sensor 11, and as a result, the temperature sensor 11 can measure the temperature with high accuracy, which is very preferable.

(e) In the present embodiment, the light receiving elements 13 are arranged at a plurality of points to surround the temperature sensor 11. When a plurality of light receiving elements 13 are arranged, it becomes possible to determine not only whether or not the position of the measurement object 3 has deviated, but also the direction of movement when the positional deviation has occurred.

Particularly, in the present embodiment, the light receiving elements 13 are arranged on all four sides when viewed from the temperature sensor 11. When the light receiving elements 13 are arranged in this way, it becomes possible to determine a general direction of the movement of the measurement object 3 in any direction.

Therefore, according to the present embodiment, by using the determination result of the moving direction of the measurement object 3, the measurement object 3 and the sensor unit 10 can be easily and quickly reset in the event of alarm processing, and further, it is also possible to alert the user of the temperature measuring device 1 by notifying the determination result of the moving direction of the measurement object 3, and as a result, the temperature measuring device 1 is extremely convenient for the user.

(f) In the present embodiment, the light emitting elements 12 are positioned between the plurality of light receiving elements 13 and arranged at a plurality of points to surround the temperature sensor 11. Therefore, according to the present embodiment, even when the light receiving elements 13 are arranged at a plurality of points, it is possible to suppress unevenness in the received light intensity of each light receiving element 13, and as a result, it becomes possible to improve a detection accuracy regarding the positional deviation of the measurement object 3.

(g) As described in the present embodiment, when the light emission from the light emitting element 12 is performed intermittently or in a mode in which the strength of the emitted light is changed, it is possible to extend the life of the light emitting element 12 while suppressing power consumption. Further, by varying a pulse frequency and adjusting a length of the light emission time, it becomes possible to control the brightness of the light from the light emitting element 12. Therefore, as a result, it is possible to contribute to the improvement of the detection accuracy regarding the positional deviation of the measurement object 3.

### 5. Modified Example

As described above, embodiments of the present invention have been specifically described. However, the present invention is not limited to the above-described embodiments, and various modifications can be made without departing from the scope of the invention.

In the present embodiment, a newborn baby or an infant housed in the incubator 2, or an animal is used as the measurement object 3, and the case of measuring the temperature (body temperature) of the measurement object 3 has been described as an example. However, the present invention is not limited thereto. That is, the present invention can be applied to the case of performing temperature measurement using the non-contact temperature sensor 11, and the measurement object 3, which is a target of the temperature measurement may be a human body other than a newborn baby or an infant, or may be an animal other than a human. Further, any object other than a living thing may be used as long as it can cause a relative positional deviation with respect to the temperature sensor 11.

Further, in the present embodiment, explanation is given for the case where the origin received light intensity is specified in response to the origin determination trigger as an example. However, the present invention is not limited thereto. For example, the origin received light intensity may be set in advance based on a device specification, an operating environment condition, etc., and stored in the storage unit 22a, without being specified in response to the origin determination trigger.

Further, in the present embodiment, explanation is given for the case where the relative positional deviation is detected with the origin received light intensity as a reference for example. However, the present invention is not limited thereto. For example, instead of using the origin received light intensity as a reference, the following case can be considered. An amount of time-series change in the received light intensity by the light receiving element 13 is monitored, and when the amount of change exceeds a predetermined threshold value, it is determined that the relative positional deviation has occurred.

Further, in the present embodiment, an arrangement mode, etc., of the light emitting element 12 and the light receiving element 13 is described with specific examples. However, these modes are merely examples and are not intended to be limited to specific modes.

### DESCRIPTION OF SIGNS AND NUMERALS

1 . . . Temperature measuring device 2 . . . Incubator 3 . . . Measurement object 10 . . . Sensor unit 11 . . . Temperature sensor 12 . . . Light emitting element 13 . . . Light receiving element 14 . . . Light emission drive circuit 15 . . . Conversion amplifier circuit 20 . . . Control unit 21 . . . Temperature measurement unit 22 . . . Deviation detecting unit 22a . . . Storage unit 22b . . . Determination unit 23 . . . Origin determination trigger unit 24 . . . Alarm processing unit 30 . . . Output unit

1. A temperature measuring device, comprising:
a temperature sensor that measures a temperature of a measurement object in a non-contact manner with the measurement object;
a light emitting element that emits light toward the measurement object;
a light receiving element that receives reflected light of the light emitted from the light emitting element; and
a deviation detecting unit that detects a relative positional deviation between the measurement object and the temperature sensor based on a received light intensity by the light receiving element.

2. The temperature measuring device according to claim 1, wherein the deviation detecting unit comprises:
a storage unit that stores a received light intensity by the light receiving element as an origin received light intensity, when the measurement object is positioned at a predetermined point; and
a determination unit that determines that a positional deviation of the measurement object has occurred when a difference between the origin received light intensity stored in the storage unit and a received light intensity obtained by the light receiving element is a predetermined threshold value or more.

3. The temperature measuring device according to claim 2, comprising an origin determination trigger unit that specifies the origin received light intensity and stores it in the storage unit.

4. The temperature measuring device according to claim 1, wherein the light emitting element and the light receiving element are attached to the temperature sensor.

5. The temperature measuring device according to claim 4, wherein at least the light receiving elements are arranged at a plurality of points to surround the temperature sensor.

6. The temperature measuring device according to claim 5, wherein the light receiving elements are arranged on all four sides when viewed from the temperature sensor.

7. The temperature measuring device according to claim 5, wherein the light emitting elements are arranged at a plurality of points to surround the temperature sensor.

8. The temperature measuring device according to claim 1, wherein the light emitting element performs light emission intermittently or in a mode in which a strength of a light emission intensity is changed.

9. A temperature measuring method, comprising:
measuring a temperature of a measurement object using a non-contact temperature sensor;
emitting light from a light emitting element toward the measurement object and receiving reflected light of the light emitted from the light emitting element, with a light receiving element; and
detecting a relative positional deviation between the measurement object and the temperature sensor based on a received light intensity by the light receiving element.
